(19) United States
(12) Patent Application Publication
(10) Pub. No.: US 2006/0165650 A1
(43) Pub. Date: Jul. 27, 2006

(54) Rantes-derived peptides with anti-hiv activity

(76) Inventors: Vincenzo Pavone, Milano (IT); Paolo Lusso, Milano (IT)

Correspondence Address: YOUNG & THOMPSON, 745 SOUTH 23RD STREET, 2ND FLOOR, ARLINGTON, VA, 22202, US

(21) Appl. No.: 10/542,857

(22) PCT Filed: Jan. 22, 2004
(86) PCT No.: PCT/IB04/00155

§ 371 Date: Jan. 6, 2006

(30) Foreign Application Priority Data
Jan. 24, 2003 (IT) M12003A000107

(51) Int. Cl. A61K 38/19; C07K 14/52

(52) U.S. Cl. 424/85.1; 530/351; 530/324

(57) ABSTRACT

RANTES-derived peptides and the use thereof in the treatment of diseases in which RANTES receptor is involved, such as viral infections, particularly HIV infections, and inflammatory, allergic, degenerative, neoplastic or metastatic diseases.

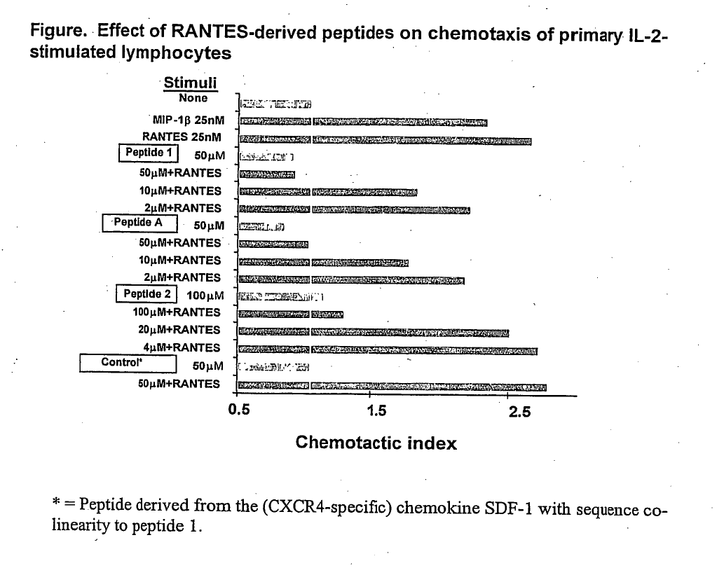

The present invention relates to peptides derived from the chemokine RANTES and the use thereof for the treatment of diseases in which RANTES and its receptors are involved. More precisely, the invention provides peptides with amino acidic sequences corresponding to the N-loop and β-1 regions of RANTES, pharmaceutical compositions containing them and the use thereof for the prevention or treatment of viral diseases, in particular AIDS, inflammatory disease such as rheumatoid arthritis or multiple sclerosis, allergic disease, degenerative disease such as arteriosclerosis, neoplastic or metastatic disease, and more generally all diseases in which chemokines or their receptors are involved.

The peptides of the invention are particularly useful for- the treatment of diseases that are related to the infection of viruses like HIV-1, other primate-lentiretroviruses (HIV-2, SIV) and other viruses which use chemokine receptors to bind the cellular surface and/or to penetrate the target cell.

### BACKGROUND OF THE INVENTION

The term chemokine is used to identify a family of chemotactic cytokines characterized by a high degree of genetic, structural and functional similarity (Immunol. Today 1993, 14:24).

Most known chemokines are grouped in two main families referred to as C-X-C and C-C, depending on the configuration of a conserved motif of two cysteine in their sequence (Ann. Rev. Immunol. 1994, 55:97-179).

Chemokines are important mediators of the inflammatory response which act through the recruitment of specific cellular populations of the immune system in the inflammatory site; the C-X-C chemokines are generally active on neutrophilic granulocytes while the C-C, chemokines are active on eosinophilic and basophil granulocytes, on limphocytes and monocytes.

RANTES, MIP-1α and MIP-1β are C-C chemokines which have been proposed as possible mediators of autoimmune and allergic diseases.

A specific antiviral effect against primate lentiretrovirus has been described for those three chemokines (Science, 1995, 270:1811-1815).

RANTES is the most potent among C-C chemokines which inhibit the HIV infection. This chemokine binds to the CCR5 receptor, which is the main membrane co-receptor for HIV-1, being it is used by most viral strains present in the population, preferably those sexually transmitted. Said receptor is therefore a primary target for possible therapeutical strategies, above all during the asymptomatic phase of HIV disease. However, the therapeutic use of natural chemokines is hampered by their pro-inflammatory activity, in that most chemokines are involved in leukocyte recruitment at the inflammation and infection sites, and in their functional activation.

A number of recent studies have suggested that an element crucial for the chemokine-induced receptor activation is located at the molecule's NH₂ terminus (J. Biol. Chem. 1991; 266:23128-23134; Biochem. Biophys. Comm. 1995, 211:100-105). Actually, a preliminary study (Nature, 1996, 383:400) and a more detailed study (Science; 1997, 276-282), both recently published, have shown that RANTES-chemokine analogues modified at the NH₂ terminus (through the deletion of 8 amino acids, or by covalently binding a complex chemical radical [amino-oxy-pentane or AOP], respectively) maintain the anti-HIV activity even though they do not induce chemotaxis in vitro or they induce it to a very low extent.

Peptides corresponding to the sequences, 7-68 to 10-68 of RANTES are disclosed in WO97/44462. RANTES mutants such as Leu-RANTES and Met-RANTES are disclosed in WO96/17935 and WO98/13495. As a matter of fact, however, the therapeutic use of small molecules or peptides is preferred, compared with the full-length protein also in the recombinant form, for a number of reasons, such as easiness of synthesis and possibility of minimizing any side-effects caused by the molecule regions which are not useful or even harmful. A number of examples of peptides in the pre-clinical phase for anti-HIV therapy can be found in literature: 1) Judice et al., PNAS 94:13426, 1997, disclose structurally rigid peptides deriving from the gp41 sub-unit of HIV envelope, which inhibit the fusion of the cell membrane; 2) Prieto et al., AIDS Res Hum Retroviruses 12:1023, 1996, disclosed modified peptides (benzyl-conjugated) deriving from CD4; or 3) Robinson et al., J Leuk Biol 63:94, 1998, disclose the activity of Indolicin 13-mer, a natural peptide of bovine origin, neutrophil and capable of inhibiting HIV virus at doses comprised from 60 to 100 μM; 4) Heveker et al., Curr Biol 8:369, 1998: describe peptides deriving from the N-terminus of SDF-1, capable of inhibiting HIV and which also lack the pro-inflammatory activity when the first two amino acids are deleted.

WO99/11666 discloses RANTES derivatives modified at the amino-terminus and the use thereof as anti-inflammatory agents in the treatment of asthma, allergic rhinitis, atopic dermatitis, atheroma/atherosclerosis, rheumatoid arthritis and as antiviral agents in the treatment of HIV infection.

WO98/51705 discloses peptides corresponding to the protein domain included between the second and the third cysteine of chemokines of the CC family, including RANTES, and the use thereof for the treatment of HIV-1 and lentivirus infections, or for the treatment of allergic or autoimmune diseases.

WO00/27880 discloses peptides derived from the sequence 10-34 of RANTES having inhibiting activity against HIV, and antiallergic and antinflammatory activities.

### DISCLOSURE OF THE INVENTION

The present invention provides peptides containing 10 to 19 amino acidic residues, of general formula (I) (unless otherwise specified, the one-letter amino acid code is used):
Ac-C-x1-PYI-x2-x3-Y-NH2 (I)
wherein Ac- represents an acetyl residue, —NH2 a carboxamido terminus, x1 and x3, which can be the same or different, are selected from the group consisting of hydrophobic residues Phe, Tyr, 1Nal (L-beta-1-naphthyl-alanine), 2Nal (L-beta-2-naphthyl-alanine), Cha (L-beta-cyclohexyl-alanine), x2 is a spacer containing 2 to 12 amino acidic residues.

According to preferred embodiments, the spacer x2 is selected from 1) the sequence 16-27 of RANTES (the reference sequence is found-in: T J Schall et al. A human T cell-specific molecule is a member of a new gene family. J. Immunol. 141, 1018-1025, 1988), 2) the sequence 16-27 of RANTES in which 1 to 3 amino acidic residues are replaced with different natural or non natural amino acids of L or D configuration, 3) the sequence 16-27 of RANTES in which any group of at least two, preferably at least three and no more than nine residues, either consecutive or non consecutive, is removed. More preferably, x2 is selected from ARPLPR-X-HIKEYF, ARPLPR-X-HIKEY1Nal, ARPLPR-X-HIKEY2Nal, ARPLPR-X-HIKEYCha, ARPLPR-X-HIF, ARPLPR-X-HYF, ARPLPR-X-EYF, ARPLPRKEYF, ARPLPIKEYF, ARP-X-HIKEYF, wherein X is Ala or Pro.

The present invention also include dimers of the peptides of formula (I), obtainable by formation of a disulfide bridge between two Cys motifs at position 1.

The compounds of the invention can be synthesized with techniques known in literature, see for example Schroeder et al., “The Peptides” vol 1, Academic Press, 1965; Bodanszky et al., “Peptide Synthesis” Interscience Publisher, 1966; Barany & Merrifield, “The peptides; Analysis, Synthesis, Biology”, 2, Chapter 1, Academic Press, 1980. These techniques include peptide synthesis in solid phase, peptide synthesis in solution, organic chemistry synthetic procedures, or any combination thereof. The selected synthetic scheme will of course depend on the composition of the specific molecule.

Preferably, synthetic procedures based on appropriate combinations of techniques in solid phase and of conventional methods in solution will be used, which involve low production, costs particularly on the industrial scale. In more detail, said procedures consist of:

i) Synthesis in solution of fragments of the peptide chain through the successive coupling of N-protected amino acids, suitably activated, with an amino acid or a C-protected peptide chain, recovery of the intermediates, successive selective deprotection of the N and C-terminus of said fragments and coupling of them until obtaining the desired peptide. Finally, when necessary, the side chains are deprotected.

ii) Synthesis in solid phase of the peptide chain from the C-terminus towards the N-terminus on an insoluble polymeric support. The peptide is removed from the resin by hydrolysis with anhydrous hydrofluoric acid or with trifluoroacetic acid in the presence of suitable scavengers, with the concomitant deprotection of the side chains.

The following sequences are particularly preferred:

| | | | | |
|---|---|---|---|---|
| Ac-CF | PYI | ARPLPRAHIKEYF | Y | -nh2 |
| Ac-CF | PYI | ARPLPRPHIKEYF | Y | -nh2 |
| Ac-C1Nal | PYI | ARPLPRAHIKEYF | Y | -nh2 |
| Ac-C2Nal | PYI | ARPLPRAHIKEYF | Y | -nh2 |
| Ac-CCha | PYI | ARPLPRAHIKEYF | Y | -nh2 |
| Ac-CF | PYI | ARPLPRPHIKEY1Nal | Y | -nh2 |
| Ac-CF | PYI | ARPLPRPHIKEY2Nal | Y | -nh2 |
| Ac-CF | PYI | ARPLPRPHIKEYCha | Y | -nh2 |
| Ac-CF | PYI | ARPLPRAHI---F | Y | -nh2 |
| Ac-CF | PYI | ARPLPRAH---YF | Y | -nh2 |
| Ac-CF | PYI | ARPLPRA---EYF | Y | -nh2 |
| Ac-CF | PYI | ARPLPR---KEYF | Y | -nh2 |
| Ac-CF | PYI | ARPLP---IKEYF | Y | -nh2 |
| Ac-CF | PYI | ARP---AHIKEYF | Y | -nh2 |
| Ac-CF | PYI | A---PRAHIKEYF | Y | -nh2 |

Peptides (I) can also be inserted in or bound to sequences of physiological proteins, such as human albumin or the fragment Fcγ of human immunoglobuline IgG, which act as non-toxic carriers for the antiviral domain, or can be bound to polyethylene glycol molecules to reduce their immunonogenicity, increase their resistance to proteolytic enzymes and improve their bioavailability. The techniques for the preparation of PEG-derivatives are known in the art and are described for example in Journal of Biological Chemistry 252(11):3578-3581 (1977), Bioconjugate Chemistry 6:150-165 (1995), U.S. Pat. No. 5,122,614, U.S. Pat. No. 5,739,208.

The peptides of the invention have been tested for their inhibitory activity against HIV virus-1 and their chemotactic activity.

The experimental details are reported in the examples. The results of the chemotaxis tests are shown in the annexed Figure.

ID₅₀ values (mean of three independent experiments, expressed as micromolar values) obtained in a HIV-1 inhibition assay, are reported in Table 1:

| | | | |
|---|---|---|---|
| | | MW | ID50 |
| Pept. | Sequence | (uma) | (μM) |

| | | | | |
|---|---|---|---|---|
| 1 | Ac-CF AYI ARPLPRAHIKEYF | Y -nh2 | 4852 | 2.5 |
| 2 | 2HN-CF PYI ARPLPRAHIKEYF | Y -nh2 | 4768 | 6.0 |
| 3 | RANTES (des 1-8) | | | 0.2 |
| A | Ac-CF PYI ARPLPRAHIKEYF | Y -nh2 | 4852 | 0.5 |
| B | Ac-CF PYI ARPLPRPHIKEYF | Y -nh2 | 4902 | 0.6 |
| C | Ac-C1Nal PYI ARPLPRAHIKEYF | Y -nh2 | 5000 | 1.5 |
| D | Ac-C2Nal PYI ARPLPRAHIKEYF | Y -nh2 | 5000 | 1.7 |
| E | Ac-Ccha PYI ARPLPRAHIKEYF | Y -nh2 | 4916 | 0.9 |
| F | Ac-CF PYI ARPLPRPHIKEY1Nal | Y -nh2 | 5004 | 0.44 |
| G | Ac-CF PYI ARPLPRPHIKEY2Nal | Y -nh2 | 5004 | 0.88 |
| H | Ac-CF PYI ARPLPRPHIKEYCha | Y -nh2 | 4916 | 0.51 |
| I | Ac-CF PYI ARPLPRAHI---F | Y -nh2 | 4012 | 1.9 |
| L | Ac-CF PYI ARPLPRAH---YF | Y -nh2 | 4112 | 0.87 |
| M | Ac-CF PYI ARPLPRA---EYF | Y -nh2 | 4096 | 0.68 |
| N | Ac-CF PYI ARPLPR---KEYF | Y -nh2 | 4210 | 0.78 |
| O | Ac-CF PYI ARPLP---IKEYF | Y -nh2 | 4124 | 0.44 |
| P | Ac-CF PYI ARP---AHIKEYF | Y -nh2 | 4120 | 0.63 |
| Q | Ac-CF PYI A---PRAHIKEYF | Y -nh2 | 4120 | 1.9 |

As shown in Table 1, the peptides of the invention (A-Q) have biological activity higher than or comparable to that of the two reference peptides, namely Ac-RANTES₁₁₋₂₉ and RANTES₁₁₋₂₉ (respectively peptides 1 and 2 in Table 1). Albeit showing biological activity slightly lower than the parent peptide of the series—RANTES (des 1-9) (peptide 3)—. The peptides of the invention (A-Q), unlike RANTES (des 1-9), can be synthetically prepared on an industrial scale with low production costs.

The experimental results suggest the importance of the following structural characteristics, as far as the peptides of the invention are concerned:
presence of the C-x1-PYI group and of x3-Y terminal residues and the possibility to modify the “linker” region with respect to the number and type of amino acids;
compared to natural RANTES and to RANTES (des 1-9), substitution of alanine with proline at position 13 (corresponding to position 3 in peptides A-Q). This substitution decreases by approx. one order of magnitude the 50%-active concentration of the peptide, which changes from about 2.5 μg/ml to about 0.4 μg/ml in HIV-mediated fusion assay, while keeping the RANTES-induced lymphocytic chemotaxis-inhibiting activity at doses similar to those effective against HIV.
acetylation of the first residue (Cys).

Furthermore, it has surprisingly been found that peptides (I) in the dimeric form exert higher anti-HIV action than their monomeric homologues.

The peptides of the invention are unable to induce activation of the CCR-5 receptor and therefore they do not cause toxic pro-inflammatory effects.

The peptides of the invention or the derivatives thereof, particularly the dimers, can be used for the therapy or the prophylaxis of AIDS and of other diseases which are caused by the infection of primate lentiretrovirus and of other viruses which utilize chemokine receptors as membrane receptors. The peptides of the invention can also be used for the treatment of allergic or autoimmune diseases, or for the treatment of tumors—as regards the role of chemokines in tumors and metastasis, see Payne A S et al., J Invest Dermatol June 2002; 118(6):915-22—or any other disease in the pathogenesis and clinical symptoms of which the chemokines are involved. The peptides of the invention will be administered, suitably formulated in pharmaceutical compositions, for example as reported in “Remington's Pharmaceutical Sciences Handbook”, Mack Publishing Company, New York, U.S.A.

The compositions will contain an effective amount of peptides (or derivatives or dimers thereof), for instance from 0.1 to 100 mg of peptide, and they will be administered preferably by the parenteral route, in particular by the subcutaneous or intramuscular routes. The daily amount will obviously depend on different factors, including severity of the disease, weight, sex and age of the patient, and it will be determined on the basis of the toxicological, pharmacokinetic and pharmacodynamic properties of each single peptide or derivative thereof. Usually the peptide daily dosage will be comprised between 10 and 1500 μmol per kg of body weight and the treatment will be maintained for a long time. Also other administration routes can be employed, for example the oral route using liposome formulations or other techniques known for the administration of peptides or proteins by the gastro-enteric route, as described in WO93/25583.

### EXAMPLE 1

Synthesis of the peptide Ac-Cys-Phe-Pro-Tyr-Ile-Ala-Arg-Pro-Leu-Pro-Arg-Ala-His-Ile-Lys-Glu-Tyr-Phe-Tyr-NH2.

This compound was prepared by peptide synthesis in solid phase. In particular, the methodology which makes use of the α-amino-protecting Fmoc group was applied. The synthesis was carried out using an automatic peptides synthesizer which operates in continuous flow, and using a solid support which provides the peptide as C-terminus amide. Furthermore, a 0.2 mmol synthetic scale with a resin substitution equivalent to 0.50 mmol/g was used. The α-amino-protecting Fmoc group of each residue, after coupling, was removed by means of a 20% by volume solution of piperidine in DMF. Two successive treatments of 3 and 7 minutes, respectively, were carried out for each cycle. Amino acids were bound in successive steps, using conventional the conditions and methodologies.

After completion of the synthesis the N-terminus was acetylated by treatment with 10 ml of a 20% by volume acetic anhydride solution in DMF for 20 minutes. Removal of the peptide from the resin and the simultaneous removal of the side chain protecting groups, were carried out by means of an ethanedithiol/anisole/TFA mixture in a 0.25/0.25/9.5 ratio (by volume) at 0° C. for 2 h. The resin was filtered, and the crude peptide was precipitated from the acidic solution with ethyl ether. 0.204 g of crude product were obtained in the form of powder. A 42% yield was obtained, based on the resin substitution degree. The analytical HPLC showed product homogeneity with a single main peak at rt=2.747. The crude material was purified by preparative RP-HPLC, obtaining 0.038 g of pure product. The purity of the product was confirmed by analytical HPLC, and the expected molecular weight of 2426 uma was confirmed by MALDI-TOF mass spectrometry.

The dimeric form was prepared with the following procedure. 10 mg of the freeze-dried monomeric form were dissolved with 2 ml of a 50% dimethyl sulfoxide aqueous solution at pH 5-6 (with 10% ammonium bicarbonate) and kept under stirring for 12 hours. Formation of the dimer was quantitative. Analytic reversed phase HPLC showed the appearance of a higher retention time component and the disappearance of the lower retention time component (Phenomenex Jupiter 5μ C18 300A column, 150×4.60 mm, 1.2 ml/min, with a gradient of 42% to 80% of aqueous acetonitrile containing 0.1% TFA during a period of 8 min). In these experimental conditions, the retention time of the starting reduced form is 2.747 min and that of the oxidized form is 4.340 min. The reaction mixture is diluted with 4 volumes of water, buffered with phosphoric acid (final concentration 50 mM) and purified by semi-preparative HPLC. The identity of the product was confirmed by MALDI-TOF mass spectrometry, which confirmed the expected molecular weight of 4847.6 uma.

### EXAMPLE 2

Synthesis of the dimeric peptides:
Ac-CFPYIARPLPRAHIKEYFY-nh2, in which
x1=F, x2=ARPLPRAHIKEY, x3=F
Ac-CFPYIARPLPRPHIKEYFY-nh2, in which
x1=F, x2=ARPLPRPHIKEY, x3=F
Ac-C1NalPYIARPLPRAHIKEYFY-nh2, in which
x1=1Nal, x2=ARPLPRAHIKEY, x3=F
Ac-C2NalPYIARPLPRAHIKEYFY-nh2, in which
x1=2Nal, x2=ARPLPRAHIKEY, x3=F
Ac-CChaPYIARPLPRAHIKEYFY-nh2, in which
x1=Cha, x2=ARPLPRAHIKEY, x3=F
Ac-CFPYIARPLPRPHIKEY1NalY-nh2, in which
x1=F, x2=ARPLPRPHIKEY, x3=1Nal
Ac-CFPYIARPLPRPHIKEY2NalY-nh2, in which
x1=F, x2=ARPLPRPHIKEY, x3=2Nal
Ac-CFPYIARPLPRPHIKEYChaY-nh2, in which
x1=F, x2=ARPLPRPHIKEY, x3=Cha
Ac-CFPYIARPLPRAHIFY-nh2, in which x1=F, x2=ARPLPRAHI, x3=F
Ac-CFPYIARPLPRAHYFY-nh2, in which x1=F, x2=ARPLPRAHY, x3=F
Ac-CFPYIARPLPRAEYFY-nh2, in which x1=F, x2=ARPLPRAEY, x3=F
Ac-CFPYIARPLPRKEYFY-nh2, in which x1=F, x2=ARPLPRKEY, x3=F
Ac-CFPYIARPLPIKEYFY-nh2, in which x1=F, x2=ARPLPIKEY, x3=F
Ac-CFPYIARPAHIKEYFY-nh2, in which x1=F, x2=ARPAHIKEY, x3=F
Ac-CFPYIAPRAHIKEYFY-nh2, in which x1=F, x2=APRAHIKEY, x3=F

The peptides described in this example were prepared with a procedure similar to that reported in Example 1. The analytical characteristics are shown in Table 2:

| Compound | Rt | Mw |
|---|---|---|
| Ac-CFPYIARPLPRPHIKEYFY-nh2 | (1) 4.10 | 4900 |
| Ac-C1NalPYIARPLPRAHIKEYFY-nh2 | (1) 5.70 | 4998 |
| Ac-C2NalPYIARPLPRAHIKEYFY-nh2 | (1) 5.87 | 4998 |
| Ac-CChaPYIARPLPRAHIKEYFY-nh2 | (1) 5.22 | 4914 |
| Ac-CFPYIARPLPRPHIKEY1NalY-nh2 | (2) 9.35 | 4972 |
| Ac-CFPYIARPLPRPHIKEY2NalY-nh2 | (2) 9.62 | 4972 |
| Ac-CFPYIARPLPRPHIKEYChaY-nh2 | (2) 8.74 | 4884 |
| Ac-CFPYIARPLPRAHIFY-nh2 | (2) 9.19 | 4007 |
| Ac-CFPYIARPLPRAHYFY-nh2 | (2) 8.97 | 4107 |
| Ac-CFPYIARPLPRAEYFY-nh2 | (2) 8.30 | 4091 |
| Ac-CFPYIARPLPRKEYFY-nh2 | (2) 8.73 | 4204 |
| Ac-CFPYIARPLPIKEYFY-nh2 | (2) 9.26 | 4121 |
| Ac-CFPYIARPAHIKEYFY-nh2 | (2) 8.54 | 4115 |
| Ac-CFPYIAPRAHIKEYFY-nh2 | (2) 8.71 | 4115 |

Reverse phase HPLC analysis conditions: Rt (1) Phenomenex Jupiter 5μ C18 300A, 150×4.60 mm 5μ, 1.2 ml/min, gradient 8 min 42% aqueous acetonitrile, 0.1% trifluoroacetic acid to 80% aqueous acetonitrile, 0.1% Tfa; Rt (2) gradient 10 min 26% to 80% aqueous acetonitrile 0.1% Tfa.

The molecular weights (Mw) were determined by MALDI-TOF mass spectrometry.

### EXAMPLE 3

HIV-1 Inhibition Assays

The HIV-1-mediated cell fusion assay was performed using a modification of the test originally developed by Berger and coworkers, based on vaccinia technology. In the modified assay, high-level expression of the HIV-1 envelope on effector cells is achieved by chronic infection of a susceptible cell line with HIV-1 rather than by infection with a recombinant vaccinia virus expressing the envelope gene. Briefly, effector cells (PM1_{Bal} or Molt-3_{IIIB}) were infected with vaccinia recombinant vTF-7.3, encoding the bacteriophage T7 RNA polymerase; in parallel, target cells (uninfected PM1 or Molt-3) were infected with vaccinia recombinant vCB-21R, containing the E. coli LacZ gene linked to the T7 promoter. The multiplicity of infection was 10 for each vaccinia recombinant. Vaccinia-infected cells were resuspended at 5×10⁵ cells/ml in modified Eagle's medium supplemented with 2.5% FBS (MEM-2.5) and incubated overnight at 32° C. At 18 h post-vaccinia infection, the cells were washed and resuspended in MEM-2.5 for the fusion assay. Effector and target cells (each at 1×10⁵/well) were mixed in 96-well plates and incubated for 2 h at 37° C.; the cells were then lysed by addition of NP-40 (0.5% final), and the β-galactosidase activity in the detergent lysates was quantitated.

Lymphocyte Chemotaxis

The chemotactic activity of chemokines and peptides was assayed in duplicate 24-well Transwell™ chambers, using 5 μm pore-size polycarbonate filter membranes (Costar). Human lymphocytes were obtained from PBMC obtained from healthy donors by cultivation in RPMI medium supplemented with 10% fetal bovine serum in the presence of interleukin-2 (100 U/ml), without prior in vitro mitogenic activation. Expression of CCR5 was monitored at 2-day intervals and the cells were analysed for chemotaxis between day 7 and day 14, when the level of CCR5 was high on the near totality of the cells. For testing the agonistic activity, chemokines or peptides were diluted in RPMI medium containing 0.3% human serum albumin and added to the lower chamber in a total volume of 500 ml; a total of 1.5×10⁵ cells in 250 ml of complete medium was added to the upper chamber. For testing the antagonistic activity, the peptides were mixed with the cells prior to addition to the upper chamber. After incubation for 2 h at 37° C., the upper chambers were removed, their bottom was carefully rinsed, and the number of cells migrated into the lower chamber was counted using a FacScan analyser at a flow rate of 60 ml/min for 40 sec. Appropriate gating on the forward and side scatters was used to exclude dead cells. The chemotactic index was calculated as the ratio between the number of cells migrated in the presence of stimuli and those spontaneously migrated in the absence of exogenous factors.

1. RANTES-derived peptides containing 10 to 19 amino acids, of formula <BR> (I) :<BR> Ac-C-x1-PYI-x2-x3-Y-NH2 (I)<BR> wherein Ac-represents an acetyl residue, —NH2 a carboxamido terminus, x1 and x3, which can be the same or different, are selected from the group consisting of the hydrophobic residues Phe, Tyr, 1Nal (L-beta-l-naphthyl-alanine), 2Nal (L-beta-2-naphthyl-alanine), Cha (L-beta-cyclohexyl-alanine), x2 is a spacer containing 2 to 12 amino acidic residues.

2. Peptides as claimed in claim 1, wherein x2 is selected from 1) the sequence 16-27 of RANTES, 2) the sequence 16-27 of RANTES in which 1 to 3 amino acidic residues are replaced with different natural or non natural L or D amino acids, 3) the sequence 16-27 of RANTES in which a group of at least two, preferably at least three and no more than nine residues, either consecutive or non consecutive, is removed.

3. Peptides as claimed in claim 1, in which x2 is selected from ARPLPR-X-HIKEYF, ARPLPR-X-HIKEYlNal, ARPLPR-X-HIKEY2Nal, ARPLPR-X-HIKEYCha, ARPLPR-X-HIF, ARPLPR-X-HYF, ARPLPR-X-EYF, ARPLPRKEYF, ARPLPIKEYF, ARP-X-HIKEYF, wherein X is Ala or Pro.

4. Dimers of the peptides (I) in which two peptides are linked by disulfide bond between Cys residues at position 1.

5. Pharmaceutical compositions containing as active ingredient a peptide (I) or a dimer thereof as claimed in the above claims.

6. Pharmaceutical compositions as claimed in claim 5, for use in the treatment of diseases in which the RANTES receptor is involved.

7. Method for the preparation of a medicament useful for the treatment of viral infections, particularly HIV infections, and of inflammatory, allergic, degenerative, neoplastic or metastatic diseases, which comprises using an effective amount of the peptides or dimmers thereof as claimed in claim 1.
